SEQUENCE LISTING (7 sequences)

SEQ ID NO: 1 - DNA, 75 bp, Artificial Sequence
  Synthetic target oligo.
tgaccagtgctatcaatcggcggagctcaaaacaaaagaaaagaggaggaaagaccggaattgcagtcatgattg

SEQ ID NO: 2 - DNA, 65 bp, Artificial Sequence
  Synthetic linker oligonucleotide.
gtgtgtgtgtgtgtgtgtgtgtgtgtgtgtcaatcatgactgcaattccggtctttcctcctctt

SEQ ID NO: 3 - DNA, 60 bp, Artificial Sequence
  Synthetic linker oligonucleotide.
ttgagctccgccgattgatagcactggtcaaaaaaaaaaaaaaaaaaaaaaaaaaaaaaa

SEQ ID NO: 4 - DNA, 80 bp, Artificial Sequence
  Synthetic target sequence.
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 30 bp, Artificial Sequence
  Synthetic sequence.
tttttttttttttttttttttttttttttt

SEQ ID NO: 6 - DNA, 10 bp, Artificial Sequence
  Synthetic sequence.
aaaaaaaaaa

SEQ ID NO: 7 - DNA, 30 bp, Artificial Sequence
  Synthetic sequence.
aaaaaaaaaaaaaaaaaaaaaaaaaaaaaa

(19) United States
(12) Patent Application Publication
(10) Pub. No.: US 2010/0035243 A1
(43) Pub. Date: Feb. 11, 2010

(54) Ultra-sensitive detection of analytes

(75) Inventors: Uwe R. Muller, Waukegan, IL (US); Phil Lefebvre, Lincolnshire, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc., Northbrook, IL (US)

(21) Appl. No.: 11/643,033

(22) Filed: Dec. 20, 2006

Related U.S. Application Data
(60) Provisional application No. 60/819,766, filed on Jul. 10, 2006.

(51) Int. Cl. C12Q 1/68; G01N 33/53

(52) U.S. Cl. 435/6; 435/7.1

(57) ABSTRACT

The present invention relates to screening methods, compositions, and kits for detecting for the presence or absence of one or more target analytes, e.g. biomolecules, in a sample. In particular, the present invention relates to methods that utilize nanoparticle probes in an in-solution homogeneous assay system for high-sensitivity detection of target proteins or nucleic acids based on flow analysis of single particles.

This application claims the benefit of priority to U.S. Provisional Application Ser. No. 60/819,766, filed Jul. 10, 2006, which is incorporated by reference in its entirety.

### FIELD OF THE INVENTION

The present invention relates to a screening method for detecting for the presence or absence of one or more target analytes, e.g., proteins, nucleic acids, or other compounds in a sample. In one application, the present invention utilizes nucleic acid reporter markers as biochemical barcodes in combination with metallic nanoparticles for detecting through measuring the shifts in resonance frequency of one or more analytes in a solution with a flow-based (flow cytometry or micro-capillary) method.

### BACKGROUND OF THE INVENTION

The detection of analytes is important for both molecular biology research and medical applications. Diagnostic methods based on fluorescence, mass spectroscopy, gel electrophoresis, laser scanning and electrochemistry are now available for identifying a variety of protein structures.¹⁻⁴ Antibody-based reactions are widely used to identify the genetic protein variants of blood cells, diagnose diseases, localize molecular probes in tissue, and purify molecules or effect separation processes.⁵ For medical diagnostic applications (e.g. malaria and HIV), antibody tests such as the enzyme-linked immunosorbent assay, Western blotting, and indirect fluorescent antibody tests are extremely useful for identifying single target protein structures.^{6,7} Rapid and simultaneous sample screening for the presence of multiple antibodies would be beneficial in both research and clinical applications. However, it is difficult, expensive, and time-consuming to simultaneously detect several protein structures under assay conditions using the aforementioned related protocols.

Polymerase chain reaction (PCR) and other forms of target amplification have enabled rapid advances in the development of powerful tools for detecting and quantifying DNA targets of interest for research, forensic, and clinical applications.²⁶⁻³² The development of comparable target amplification methods for proteins could dramatically improve medical diagnostics and the developing field of proteomics.³³⁻³⁶ Although one cannot yet chemically duplicate protein targets, it is possible to tag such targets with oligonucleotide markers that can be subsequently amplified with PCR and then use DNA detection to identify the target of interest.³⁷⁻⁴⁵ This approach, often referred to as immuno-PCR, allows one to detect proteins with DNA labels in a variety of different formats. To date, all immuno-PCR approaches involve heterogeneous assays, which involve initial immobilization of a target analyte to a surface with subsequent detection using an antibody with a DNA label (for example, see U.S. Pat. Nos. 5,635,602, and 5,665,539). The DNA label is typically strongly bound to the antibody (either through covalent interactions or streptavidin-biotin binding).

Although these approaches are notable advances in protein detection, they have several drawbacks: 1) limited sensitivity because of a low ratio of DNA identification sequence to detection antibody; 2) slow target binding kinetics due to the heterogeneous nature of the target capture procedure, which increases assay time and decreases assay sensitivity; 3) complex conjugation chemistries that are required to chemically link the antibody and DNA-markers; and 4) require a PCR amplification step.⁴⁵ Therefore, a sensitive, and rapid method for detecting target analytes in a sample that is amenable to multiplexing and easy to implement is needed.

For DNA detection methods, many assays have been developed using radioactive labels, molecular fluorophores, chemiluminescence schemes, electrochemical tags, and most recently, nanostructure-based labels.⁶¹⁻⁷⁰ Although some nanostructure-based methods are approaching PCR in terms of sensitivity, none thus far have achieved the 1-10 copy sensitivity level offered by PCR. A methodology that allows for PCR-like signal amplification without the complexity, expense, and time and labor intensive aspects associated with PCR would provide significant advantages over such PCR-based methods. Methods of synthesizing unique nanoparticle-oligonucleotide conjugates are well known, for example, in U.S. Pat. Nos. 6,750,016 and 6,506,564, which are hereby incorporated in their entirety. Previously, a method has been disclosed that utilizes reporter oligonucleotides as biochemical barcodes for detecting one or more analytes in a solution, as described in U.S. patent application Ser. No. 11/127,808, and International Patent Application Nos. PCT/US05/16545, filed May 12, 2005 and PCT/US04/20493, filed Jun. 25, 2004, which are hereby incorporated by reference in its entirety.

Current techniques cover the shift in the frequency of scattered light as a consequence of target-mediated nanoparticle aggregation.⁷¹ However, conventional photometric techniques are not sensitive enough to detect low target quantities (e.g. less than attomolar levels) in bulk experiments. Detection of single binding events have been reported using microscopy,⁷² but this method is presently hampered by low throughput and is not amenable to automation. The biobarcode assay, such as that disclosed in U.S. patent application Ser. No. 11/127,808, provides high sensitivity but is limited in throughput due to the need for detection of barcodes by hybridization on a slide. Flow cytometry is a means of detecting rare micron-sized cells or particles in large populations, and has become adapted for high-throughput clinical screening (e.g. 24-tube and 96-well samplers). Combining the above techniques could offer a new means of rapidly and sensitively detecting barcodes or non-amplified targets via nanoparticle aggregation in a clinically applicable format.

Conventional wisdom in flow cytometry holds that the signal from particles much smaller than one micrometer would be lost in the signal from sample debris and electronic noise and thus remain undetectable. However, the intensity of noble metal nanoparticle plasmon resonance scatter has been reported to be significantly higher than the fluorescence yield from standard fluorophores,⁷³⁻⁷⁴ suggesting that they should be detectable by flow cytometry. Previous work has shown that gold nanoparticles can be used as labels to make cells,⁷⁵⁻⁷⁶ or microparticles⁷⁷ detectable by flow analysis. However, there have been no reports that describe detection of individual nanoparticles by flow cytometry. The preliminary experiments shown below confirm the hypothesis that individual nanoparticles can be detected by this technique, opening new avenues for molecular diagnostics.

A variety of novel bar coding systems have been developed as multiplex testing platforms for applications in biological, chemical and biomedical diagnostics. Instead of identifying a target through capture at a specific locus on an array, target analytes are captured by a bar coded tag, which then uniquely identifies the target akin to putting a UPC barcode on a product. This requires an appropriate surface functionalization to ensure that the correct target is captured with high efficiency. Moreover the tag, or barcode, has to be readable with minimal error and at high speed, typically by flow analysis. For quantitative assays the target may be labeled separately, or the tag may also serve as the label. A great variety of materials and physico-chemical principles have been exploited to generate a plethora of novel bar coding systems. Their advantages compared to microarray based assay platforms include in solution binding kinetics, flexibility in assay design, compatibility with microplate based assay automation, high sample throughput, and with some assay formats, increased sensitivity.

The assay platform disclosed in U.S. patent application Ser. No. 11/127,808, filed May 12, 2005, which is hereby incorporated by reference in its entirety, uses bar coded nanoparticles for signal amplification, converting a single captured target into a multiplicity of bar codes. For detection and decoding, however, the bar codes have to be first released from the nanoparticle and then recaptured by hybridization on an array and further hybridized with nanoparticle probes for detection. This process adds significant time and reduces the sensitivity of the assay, since thousands of barcodes are required to generate a detectable signal over noise. Moreover, arrays are expensive and the required silver amplification increases assay variability. The invention herein describes a detection technology that avoids these problems.

### SUMMARY OF THE INVENTION

The invention overcomes many of the problems of the prior art while greatly expanding the flexibility, adaptability and usefulness of techniques directed to the amplification of a signal to facilitate detection. The present invention relates to methods, probes, compositions, and kits that utilize binding moieties, such as oligonucleotides as biochemical barcodes, for detecting at least one specific target analyte in one solution. The approach takes advantage of recognition elements of specific binding pairs functionalized either directly or indirectly with nanoparticles, and the previous observation that hybridization events that result in the aggregation of gold nanoparticles can significantly alter their physical properties (e.g. optical, electrical, mechanical).⁸⁻¹²

It is well known in the art that metallic nanoparticles of 30 nm or larger diameter will change color when brought into close proximity. This principle has been exploited by functionalizing such nanoparticles with DNA oligonucleotides or with antibodies for the detection of either nucleic acid or protein analytes. The color shift as a function of surface plasmon resonance can be detected most sensitively by observing the frequency of the scattered light. It is further known that fluorescently stained beads, cells or particles can be detected and separated by flow analysis, for example on a flow cytometer or in a microcapillary through laser enhanced fluorescence detection. In this method, particles pass in single file by a detection window, which allows counting of single events (single cells, particles or beads). Due to the confinement of the particles into a small sample volume, isolated from other particles, the background is significantly reduced, enabling signal to background ratios that make single particles detectable. This method can therefore deliver superior sensitivities over other methods, where a signal is measured from a bulk sample. Note that there are many types of coding systems for beads or particles, including shape and size of beads, radio-frequency encoding, or chemical encoding, whereby the signal may be detected by light reflection, diffraction, scatter, or spectral analysis. For example, it is known that metallic nanoparticles can be coded with Raman active dyes that give each particle a unique Raman signature. They are most sensitively detected by surface enhanced Raman spectroscopy (SERS). See International Application No. PCT/US03/14100, filed May 7, 2003, which is incorporated by reference in its entirety.

In its current format, the biobarcode technology detects protein and nucleic acid targets through sandwiching between a magnetic bead and an amplifier nanoparticle that is coated with oligonucleotides of specific sequence (barcodes). By releasing the barcodes each captured target is converted into multiple surrogate targets, which are detected via hybridization to a microarray.

In one aspect of the present invention, the array hybridization detection of the above mentioned released barcodes is replaced with a flow based method, such as either a flow cytometer or a microcapillary. Since a minimum of 10,000 target DNA molecules (e.g. barcode molecules) are required in the hybridization solution to generate sufficient hybridization events on a single spot in the microarray to achieve a detectable signal, the flow method is up to 1000 fold more sensitive, since single hybridization events can be measured, and the counting of 10 events may provide sufficient statistical significance.

In another aspect of the present invention, target analytes can be detected directly. Analysis of protein or DNA targets by flow is faster than by capture on a slide or a microplate, as for example in a microplate-based ELISA, because is the flow-based analysis affords a homogeneous assay format (i.e. the nanoparticle probes that bind target do not have to be separated from nanoparticles that don't bind target), and because in solution hybridization kinetics are much faster than hybridizations to a solid surface. In assays where the presence of only a single target is to be measured, the target can be captured between two metallic nanoparticles, resulting in a change in the extinction characteristics of the nanoparticle probes that can be observed⁷⁹ as a color change based on measuring absorbance. Storhoff et al. had shown that this can be measured much more sensitively when measuring the scatter light.⁷¹ This concept can be exploited via flow analysis by the binding of targets between two nanoparticles. Since each nanoparticle is basically analysed in a small confined volume, it is physically separated from the other particles and therefore even a very small number of aggregates can be detected.

The combination of the biobarcode technology with the flow-based (flow cytometry or microcapillary) barcode detection method brings surprising new advantages over the existing biobarcode technology and conventional flow-based methods. For instance, it is known that more than 10,000 barcodes per assay are required to obtain a detectable signal by hybridization to a slide. Thus, assuming arguendo an amplification of 10 barcodes per captured target, one can detect about 1000 captured targets at best. However, if one could detect say 100 barcodes by flow analysis, the detection limit would improve by two logs.

There are several ways by which the barcodes can be detected in a flow system. For example, a nanoparticle of a particular size, shape, and/or composition can be used as a probe to bind to a barcode specific for a captured target analyte, thereby permitting detection of one type of target analyte in a sample. In another example, aggregation of two nanoparticles having the particular sizes, shapes, and/or compositions (e.g. two 30 nm or larger nanoparticles) can be used to bind a specific barcode. Either way, using the present invention the released barcodes do not have to be recaptured on a microarray but can now be detected directly by flow in a simple and homogeneous detection format.

The invention also provides methods for multiplex analysis (i.e. detecting multiple target analytes in a sample). In a typical multiplex analysis more than a single target is to be identified in a single assay. In the case of the biobarcode assay, multiple barcodes can be used and decoded. This can be achieved, for example, by binding the released barcodes to either a single or two or more nanoparticles, as described above, whereby each nanoparticle has a unique plasmon resonance frequency due to their specific size, shape, and/or composition. Aggregation of two particles would shift that specific frequency. In certain aspects of the invention, these methods can be combined, whereby some barcodes are detected by the unique signature of single nanoparticles, while others are detected by the unique signatures generated through aggregation of two or more particles. Thus, the unique resonance signatures of the nanoparticles would reveal which barcode is present.

A further method of multiplexing is provided by coding the nanoparticles with Raman active dyes, which can be sensitively detected and decoded in flow by surface enhanced Raman spectroscopy. The main advantage of this type of multiplexing over conventional biobarcode assays, where decoding of barcodes is achieved via hybridization to an array, is assay speed and sensitivity.

Another powerful approach to multiplexing is provided by combining the detectability of single nanoparticles with the coding power of fluorescently labeled microbeads. The microbeads can contain binding moieties as described herein, such that the microbeads can bind to either the target analyte or to the nanoparticles that are bound to the target analyte. Due to the large size of these beads they can be labeled with thousands of fluorescent molecules, providing for detectability and high coding capacity, achieved by varying the number and type of fluorophors. However, the binding of a single target analyte to such a microbead cannot be detected by conventional fluorescent labels, since that signal is too weak and would furthermore be swamped out by the fluorescence from the microbead.

However, if one of the microbeads now binds a gold nanoparticle via a captured target, then this nanoparticle can be detected by scattered light. The frequencies of fluorescent light and scatter light involved can be chosen not to overlap. It is important to note that the number of photons scattered from a 60-80 nm particle is about 1,000,000 times larger than the number of photons generated by a standard fluorophor label. Thus, a single nanoparticle can be detected, while a “barcoded” microbead would have to bind sufficient target/bead to get labeled with ˜1,000,000 fluorophors. It follows that in order to achieve this much target binding, target molecules have to be in excess of beads in the traditional bead assay, requiring up-front target amplification by PCR. The approach described in this invention would allow for detection of a very small number of targets without amplification, since the binding of a single target to a bead, followed by the binding of a single nanoparticle probe, would make this complex detectable and decodable.

In addition, barcodes or any other target can be captured by magnetic bead or other surface, and can then be labeled with a specific nanoparticle. The labeled target can be removed from the sample matrix, or the sample matrix can be washed away. The labeling particle can be released into solution and counted individually by flow based methods as described herein.

In one aspect, the invention provides a method for detecting for the presence of one or more target analytes in a sample, wherein the method comprises the steps of:
a) providing a plurality of nanoparticle probes conjugated to binding moieties capable of binding to a first binding site of the target analyte, wherein the nanoparticle probes comprise a metallic material and have an average diameter of less than 200 nanometers;
b) providing a capture surface comprising binding moieties capable of binding to a second binding site of the target analyte;
c) contacting the nanoparticle probes and capture surface with a sample believed to contain target analytes under conditions effective to allow for binding of the target analyte with the nanoparticle probes and the capture surface to form a complex in the presence of the target analyte;
d) optionally washing the capture surface containing the complex formed in (c) to remove all non-bound nanoparticle probes;
e) releasing the captured nanoparticle probes from the capture surface;
f) subjecting the released nanoparticle probes to confinement conditions under which individual nanoparticle probes can be detected;
g) irradiating nanoparticle probes in the confinement conditions with a light beam; and
h) measuring scatter light generated in step (g) to determine the number of released nanoparticle probes as an indicator of the presence of target analyte in the sample.

In another aspect, the invention provides a method for detecting for the presence of one or more target analytes in a sample comprising the steps of:
a) providing a first nanoparticle probe conjugated to binding moieties capable of binding to a first binding site of the target analyte, wherein the nanoparticle probes comprise a metallic material and have an average diameter of less than 200 nanometers;
b) providing a second nanoparticle probe conjugated to binding moieties capable of binding to a second binding site of the target analyte, wherein the nanoparticle probes comprise a metallic material and have an average diameter of less than 200 nanometers;
c) contacting the first and second nanoparticle probes with a sample believed to contain target analytes under conditions effective to allow for binding of the target analyte with the binding moieties on the first and second nanoparticle probes to form a complex in the presence of the target analyte;
d) subjecting the sample-nanoparticle probe mixture in (c) to confinement conditions under which individual nanoparticle probes or nanoparticle probe-target complexes can be detected;
e) irradiating the complex with light of a frequency range that covers the plasmon resonance frequency of the nanoparticles; and
f) measuring scatter light frequency to differentiate single from complexed nanoparticles,
whereby the presence of complexed particles is indicative of the presence of the target analyte in the sample.

In certain aspects, the nanoparticle probes bind to the target analyte indirectly via specific linker molecules.

In other aspects, the binding moieties comprise oligonucleotides, antibodies, aptamers, or some combination thereof.

In certain aspects, the nanoparticle probes are about 30 to about 150 nm in diameter.

In additional aspects, the target analyte is a protein or hapten and the binding moieties are antibodies. The antibodies can be polyclonal antibodies or monoclonal antibodies.

In further aspects, the target analyte is a sequence from a genomic DNA sample and the binding moieties are oligonucleotides, the oligonucleotides having a sequence that is complementary to at least a portion of the genomic sequence. The genomic DNA can be eukaryotic, bacterial, fungal or viral DNA. Also, the target analyte can be a sequence from episomal DNA sample and the binding moieties are oligonucleotides, the oligonucleotides having a sequence that is complementary to at least a portion of the episomal DNA sequence.

In certain aspects, the confinement conditions are generated by flow cytometry or by capillary eletrophoresis.

In other aspects, at least one of the nanoparticle probes can be further labeled with a Raman active group. The Raman active group can be used in equal or different concentrations.

In additional aspects, the nanoparticle probes comprise gold, silver, copper, or platinum, or are core-shell nanoparticles.

In yet other aspects, the target analyte can bind to the capture surface indirectly via specific linker molecules.

In other aspects, the capture surface can be a microtiter well. In some aspects, the capture surface containing the complex formed in step (c) above can be isolated from all non-bound nanoparticle probes. In such cases, the capture surface can be a magnetic bead.

In certain aspects, the plurality of nanoparticle probes used in a method of the invention comprises nanoparticle probes of different shapes, each differently shaped nanoparticle probe being conjugated to binding moieties that bind to a different target analyte, and wherein each differently shaped nanoparticle probe creates unique scatter light when irradiated, thereby indicating the presence of the target analyte to which it binds. The plurality of nanoparticle probes can comprise nanoparticle probes of different materials, each nanoparticle probe of different material being conjugated to binding moieties that bind to a different target analyte, and wherein each nanoparticle probe of different material creates unique scatter light when irradiated, thereby indicating the presence of the target analyte to which it binds. The plurality of nanoparticle probes can further comprise nanoparticle probes of different sizes, each nanoparticle probe of different size being conjugated to binding moieties that bind to a different target analyte, and wherein each nanoparticle probe of different size creates unique scatter light when irradiated, thereby indicating the presence of the target analyte to which it binds.

In some aspects, the methods of the invention further comprise a step of providing one or more labeled microbeads that can bind to either the target analyte or to a nanoparticle probe, thereby capable of forming a complex with the nanoparticle probes and the target analyte. In these aspects, at least one microbead can be fluorescently labeled.

Specific embodiments of the present invention will become evident from the following more detailed description of certain embodiments.

### DESCRIPTION OF THE FIGURES

FIG. 1 indicates that gold and silver nanoparticles of various sizes can be used to resolve differences in relative scatter, and the side scatter patterns generated by each size particle are sufficiently different to allow identification of the nanoparticle's size.

FIG. 2 demonstrates that silver staining of gold nanoparticles causes a large shift in side scatter, indicating a significant change in particle size and scatter properties.

FIG. 3 indicates that Plasmon scatter light from silver particles can be seen by flow cytometry.

FIG. 4 demonstrates that the complementary DNA-induced aggregation showed a time dependent increase in SSC and 630 nm intensity. The 630 nm scatter intensity of the aggregates were so intense as to begin to move off-scale.

FIG. 5 indicates that increased amounts of dA80 target induced a new population with higher side scatter and 630 nm scatter, presumably dT30 nanoparticle dimers.

FIG. 6 illustrates the detection of a viral target having at least a first portion and a second portion. The detection of the target is accomplished using two linker oligos (linker A and linker B), a nanoparticle probe A, and a nanoparticle probe B. Nanoparticle probe A is conjugated with at least one poly AC oligo. Linker A comprises at least a first portion and a second portion, said first portion comprising a poly GT oligo (complementary to the poly-AC oligo conjugated to Nanoparticle probe A) and said second portion comprising a sequence complementary to the first portion of the viral target. Nanoparticle probe B is conjugated with at least one poly-T oligos. Linker B comprises a first portion and a second portion, said first portion comprising a poly A oligo (complementary to the poly T oligo conjugated to Nanoparticle probe B), and said second portion comprising a sequence complementary to the second portion of the viral target. Increased amounts of the oligo target induced increased amounts of aggregates, and in the presence of constant target and probe, the fraction of aggregates increase over time.

FIG. 7 is an illustratration showing the detection of a target molecule having at least a first portion and a second portion.

FIG. 8 shows graphs illustrating the intensity changes by 535 nm laser correlate with size, similar to intensity changes induced by white light.

FIG. 9 shows graphs illustrating the white light scatter of single nanoparticles and aggregated nanoparticles as measured by Ocean Optics spectrophotometer, and dot plots showing light scatter of single nanoparticles and aggregated nanoparticles as measured by MoFlow flow cytometer.

FIG. 10 is an illustration showing a complex of methicillin resistant Staphylococcus aureus (MRSA) target with four 50 nm nanoparticle probes as described in Example 8 below.

FIG. 11 shows detection of various concentrations of MRSA target using 50 nm nanoparticle probes and flow cytometry.

FIG. 12 shows detection of prostate specific antigen (PSA) using 30 nm nanoparticle probes and flow cytometry.

### DETAILED DESCRIPTION OF THE INVENTION

Unless otherwise required by context, singular terms shall include pluralities and plural terms shall include the singular.

As utilized in accordance with the present disclosure, the following terms, unless otherwise indicated, shall be understood to have the following meanings:

The terms “target,” “analyte” or “target analyte” refer to the compound or composition to be detected, including drugs, metabolites, pesticides, pollutants, and the like. The analyte can be comprised of a member of a specific binding pair (sbp) and may be a ligand, which is monovalent (monoepitopic) or polyvalent (polyepitopic), preferably antigenic or haptenic, and is a single compound or plurality of compounds, which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. If the analyte is monoepitopic, the analyte can be further modified, e.g. chemically, to provide one or more additional binding sites. In practicing this invention, the analyte has at least two binding sites. The monoepitopic ligand analytes will generally be from about 100 to 2,000 molecular weight, more usually from 125 to 1,000 molecular weight. Typical analytes may be much larger and include, but are not limited to episomal DNA, genomic DNA, viral nucleic acid molecules, proteins, peptides, nucleic acid segments, molecules, cells, microorganisms and fragments and products thereof, or any substance for which attachment sites, binding members or receptors (such as antibodies) can be developed.

As used herein, the terms “barcode”, “biochemical barcode”, “biobarcode”, “reporter barcode” etc. are all interchangeable with each other and have the same meaning. In the preferred embodiment of the present invention, the biobarcodes are nucleic acids. The markers may be the same, or may be different. The biobarcode assay has been disclosed in U.S. patent application Ser. No. 11/127,808, filed May 12, 2005, U.S. patent application Ser. No. 10/877,750, filed Jun. 25, 2004, International Patent Application PCT/US04/020493 (Publication No. WO05/003394), filed Jun. 25, 2004, and International Patent Application PCT/US05/16545 (Publication No. WO2006/078289), filed May 12, 2005, all of which are incorporated by reference herein in their entirety.

The polyvalent ligand analytes will normally be larger organic compounds, often of polymeric nature, such as polypeptides and proteins, polysaccharides, nucleic acids, and combinations thereof. Such combinations include components of bacteria, viruses, chromosomes, genes, mitochondria, nuclei, cell membranes and the like.

For the most part, the polyepitopic ligand analytes to which the invention can be applied will have a molecular weight of at least about 5,000, more usually at least about 10,000. In the polymeric molecule category, the polymers of interest will generally be from about 5,000 to 5,000,000 molecular weight, more usually from about 20,000 to 1,000,000 molecular weight; among the protein analytes of interest, the molecular weights will usually range from about 5,000 to 200,000 molecular weight.

A wide variety of proteins may be considered as belonging to the family of proteins having similar structural features, proteins having particular biological functions, proteins related to specific microorganisms, particularly disease causing microorganisms, etc. Such proteins include, for example, immunoglobulins, cytokines, enzymes, hormones, cancer antigens, nutritional markers, tissue specific antigens, etc.

The types of proteins, blood clotting factors, protein hormones, antigenic polysaccharides, microorganisms and other pathogens of interest in the present invention are specifically disclosed in U.S. Pat. No. 4,650,770, the disclosure of which is incorporated by reference herein in its entirety.

The analyte may be a molecule found directly in a sample, such as a body fluid from a host. The sample can be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

The term “sample” as used herein refers to any quantity of a substance that may comprise target analytes, and that can be used in a method of the invention. For example, the sample can be a biological sample or can be extracted from a biological sample derived from humans, animals, plants, fungi, yeast, bacteria, viruses, tissue cultures or viral cultures, or a combination of the above. A sample may contain or be extracted from solid tissues (e.g. bone marrow, lymph nodes, brain, skin), body fluids (e.g serum, blood, urine, sputum, seminal or lymph fluids), skeletal tissues, or individual cells. Alternatively, the sample can comprise purified or partially purified nucleic acid molecules and, for example, buffers and/or reagents that are used to generate appropriate conditions for successfully performing a method of the invention. In certain embodiments, a sample is or is in solution, and can be subject to flow based detection methods as described herein.

The term “particle” as used herein specifically encompasses both nanoparticles and microparticles as defined and described hereinbelow. As used herein, the term “particle” refers to a small piece of matter that can preferably be composed of metals, silica, silicon-oxide, or polystyrene. A “particle” can be any shape, such as spherical or rod-shaped.

In certain embodiments, the methods of the invention involve the use of nanoparticle probes. Nanoparticles useful in the practice of the invention include metal (e.g., gold, silver, copper and platinum), colloidal materials. The size of the nanoparticles is preferably from about 30 nm to about 200 nm (mean diameter). The nanoparticles can be any shape, such as spherical or rod-shaped. As used herein, a “metallic” nanoparticle comprises at least one metal.

Methods of making metal nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). See also U.S. Pat. No. 6,506,564, which is incorporated by reference in its entirety.

Nanoparticles useful in the methods of the invention can also be core-shell particles such as the ones described in U.S. patent application Ser. No. 10/034,451, filed Dec. 28, 2002 and International application no. PCT/US01/50825, filed Dec. 28, 2002, which are incorporated by reference in their entirety.

Suitable nanoparticles are also commercially available from, e.g., Ted Pella, Inc. (gold), Amersham Corporation (gold), Nanoprobes, Inc. (gold).

For stability purposes, a nanoparticle probe can have zero, one, or a plurality of oligonucleotides, as well as the binding moieties, attached to it. For example, nanoparticles can be incubated with binding moieties and oligonucleotides in a 3:1 ratio. In one embodiment, the oligonucleotides are polyadenosine oligonucleotides, for example Alo, which is an oligonucleotide consisting of 10 adenosines.

Those of skill in the art will appreciate that nanoparticles can be designed to have different scatter light properties based on their size, composition, and shape. Thus, one of skill in the art can select a particular size, composition, and/or shape to represent the presence of a particular target analyte. For example, a gold, round, 30 nm nanoparticle will cause different scatter light than a silver, 60 nm, rod-shaped nanoparticle. Consequently, both probes can be used in one sample to detect the presence of two different target analytes, as discussed herein.

As used herein, the term “linker molecule” refers to a binding moiety that serves as an indirect link between a nanoparticle probe and a target analyte, or between a capture surface and a target analyte. A linker molecule can be a “linker oligonucleotide” with at least two binding regions, one of which binds to a complementary oligonucleotide conjugated to a nanoparticle or capture surface, and the other which binds to a complementary portion of the target analyte. Other examples of linker molecules include streptavidin, avidin, or antibodies. Alternatively, linkers can be generated from any of the binding moieties described below, whereby, for an example, two different moieties are chemically linked, now having specificity for two different binding partners.

The term “binding moieties” is used herein to refer to members of a specific binding pair. The term “specific binding pair (sbp) member” refers to one of two different molecules, which specifically binds to and can be defined as complementary with a particular spatial and/or polar organization of the other molecule. The members of the specific binding pair can be referred to as ligand and receptor (antiligand). These will usually be members of an immunological pair such as antigen-antibody, although other specific binding pairs such as biotin-avidin, enzyme-substrate, enzyme-antagonist, enzyme-agonist, drug-target molecule, hormones-hormone receptors, nucleic acid duplexes, IgG-protein A/protein G, polynucleotide pairs such as DNA-DNA, DNA-RNA, protein-DNA, lipid-DNA, lipid-protein, polysaccharide-lipid, protein-polysaccharide, nucleic acid aptamers and associated target ligands (e.g., small organic compounds, nucleic acids, proteins, peptides, viruses, cells, etc.), and the like are not immunological pairs but are included in the invention and the definition of sbp member. A member of a specific binding pair can be the entire molecule, or only a portion of the molecule so long as the member specifically binds to the binding site on the target analyte to form a specific binding pair.

The term “ligand” refers to any organic compound for which a receptor naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog will usually differ from the ligand by more than replacement of a hydrogen with a bond, which links the ligand analog to a hub or label, but need not. The ligand analog can bind to the receptor in a manner similar to the ligand. The analog could be, for example, an antibody directed against the idiotype of an antibody to the ligand.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, nucleic acid aptamers, avidin, protein A, barstar, complement component C1q, and the like. Avidin is intended to include egg white avidin and biotin binding proteins from other sources, such as streptavidin.

The term “specific binding” refers to the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

The term “non-specific binding” refers to the binding between molecules that is relatively independent of specific surface structures. Non-specific binding may result from several factors including hydrophobic interactions between molecules.

In certain embodiments, a label can be used to further differentiate a target analyte in a sample. For example, nanoparticle probes can serve as labels directly, or their optical properties can be modified by linkage to a Raman-active group. A “capture surface” as used herein can be any surface capable of having antibodies, aptamers, oligonucleotides, or analytes bound thereto. Such surfaces include, but are not limited to, glass, metal, plastic, or materials coated with a functional group designed for binding of antibodies, aptamers, oligonucleotides, or analytes. The coating may be thicker than a monomolecular layer; in fact, the coating could involve porous materials of sufficient thickness to generate a porous 3-dimensional structure into which the antibodies, aptamers, oligonucleotides, or analytes can diffuse and bind to the internal surfaces. Binding of antibodies, aptamers, oligonucleotides, or analytes to a substrate can be accomplished by any method known to those of skill in the art and as described, for example, in U.S. patent application Ser. No. 11/124609, filed May 6, 2005, which is incorporated by reference in its entirety.

A “capture surface” suitable for the methods of the invention include, but are not limited to, microplates, glass slides, nanoparticles, magnetic beads, or any suitable inorganic or organic molecule of sufficient size, or a combination thereof, that offers the appropriate surface for attachment of antibodies, aptamers, oligonucleotides, or analytes, and shows a minimum of non-specific binding to nanoparticle probes that are not bound to target analytes. In one embodiment, the surface is a magnetic (e.g., ferromagnetite) colloidal material. The complex formed between the nanoparticle probe, the target analyte, and the magnetic surface can be easily separated from any unbound components by the application of a magnetic field. In another embodiment, the complex can be separated by centrifugation. In certain embodiments, the magnetic surface is a magnetic bead, such as a magnetic microparticle.

In certain embodiments, a nanoparticle probe bound to target analyte forms a complex with the capture surface through binding of the target analyte to a binding moiety that is attached to the capture surface itself. Once the complex is formed, any unbound probes are removed from the complex by suitable methods, such as, without limitation, washing, centrifugation, and application of a magnetic field. The complex can be disrupted by releasing the nanoparticle can be released from a capture surface using techniques well known to those of skill in the art. For example, specifically bound probes can be selectively released from the capture surface by any suitable methods, including but not limited to, target analyte displacement, epitope displacement, antibody displacement, aptamer displacement, target analyte destruction, antibody destruction, aptamer destruction, protease digestion, restriction digestion, a reducing agent, RNaseH digestion, chemical cleavage, and dehybridization, depending on what binding moiety is used to capture the target analyte bound nanoparticle probes.

In some instances, a “detaching agent” can be used to release the capture nanoparticle probes from the capture surface. As used herein, a “detaching agent” refers to a solution or agent that can disrupt or destruct the linkage of a binding moiety to the capture surface, and detach and release the binding moiety in complex with the nanoparticle probe into solution. For example, where the binding moiety is an oligonucleotide, it can be detached and released from the capture surface by dehybridization, dissolution, or chemical cleavage. Representative detaching agents include, without limitation, iodine, a cyanide salt, and a basic agent. See also U.S. patent application Ser. No. 11/127808, and International Patent Application Nos. PCT/US05/16545, filed May 12, 2005 and PCT/US04/20493, filed Jun. 25, 2004, which are hereby incorporated by reference in its entirety.

In certain embodiments, a sample having been contacted with nanoparticle probes will be spatially confined in a sample stream under confinement conditions (such as those described in the Examples herein). As used herein, “confinement conditions” refer to the spatial arrangement of the sample in such a manner that allows for analysis of individual nanoparticle probes within the sample using flow-based methods (e.g. flow cytometry or microcapillary electrophoresis). Confinement can be accomplished using methods well known to those of skill in the art. Conventional methods involve “electrokinetic focusing,” as discussed, for example, in U.S. Pat. No. 6,120,666, which is incorporated by reference. Electrokinetic techniques include electroosmosis and/or electrophoresis. Two common types of electrophoresis are steady state and capillary zone electrophoresis as discussed by Hahm and Beskok, 2005, Bull. Polish Acad. Sci. 53:325-334.Once in confinement conditions, the sample stream can be irradiated with a light beam. The confinement conditions permit the nanoparticle probes to flow single-file past the light beam, such as a laser beam (and in many instruments, past two or more laser beams). The momentary pulse of scatter light emitted as the particle crosses the beam is measured by photomultipliers at some angle (typically 90 degree angle) from the beam. Typically, two to three detectors are used with different wavelength bandpass filters, allowing the simultaneous detection of emissions at different wavelengths from different nanoparticles, or fluorescence light from the fluorescently coded microparticles, respectively.

In addition to fluorescence, two types of light scatter are measured in traditional flow cytometry. Low-angle forward scatter (often called simply “forward scatter”) is roughly proportional to the diameter of the particle. Orthogonal, 90° or “side scatter” is proportional to the granularity. Thus, in the FACScan, each particle can provide up to five numbers: size, granularity, plus green, red, and far red fluorescence intensities.

In a dot plot, each cell is represented by a dot, positioned on the X and Y scales according to the intensities detected for that cell. Scatter dot plots (X=forward scatter intensity; Y=side scatter intensity) are often informative (see examples below). Scatter scales are usually linear. Fluorescence dot plots typically plot X=green fluorescence intensity, Y=red fluorescence intensity. These two-color dot plots are often divided into four quadrants, the double negative cells, the green-only, red-only, and double positive cells. These are quantitated by giving the percentage of cells in each quadrant. Since fluorescence intensity often varies several orders of magnitude between cells, the scales are usually the logarithm of fluorescence intensity spanning four decades (a 10,000-fold range).

Histograms are often used to interpret results of a flow-based assay. In a histogram, the X axis is intensity (of scatter or fluorescence), and the Y axis shows how many cells had each intensity. Thus, histograms show the distribution of intensities for a single parameter, while dot plots show the correlated distribution for two parameters. The density of dots in a region of a dot plot shows the “number of cells”, equivalent to the Y axis of a histogram. Indeed, dot plots are sometimes represented as pseudo-3D graphs where the Z axis is “number of cells”.

As shown in the Examples herein, it is feasible to detect scatter light from individual gold and silver nanoparticles using a standard flow cytometer, and distinguish between different sizes and types of nanoparticles. More importantly, changes in nanoparticle scatter induced by several different phenomena can also be detected and differentiated. Most notably, the change in red scatter of 60 nm Au complementary DNA binding-induced aggregation was sufficiently high to make these nanoparticle aggregates detectable and countable. Therefore, the aggregation of two nanoparticles which are brought together via binding by linker oligonucleotides to a target oligonucleotide can be detected using flow cytometry. Aggregated nanoparticles formed very bright and tight scatter profiles, making them easy to detect, differentiate from nanoparticle monomers, and quantitate.

Numerous parameters make themselves amenable to nanoparticle detection, permitting those of skill in the art to design sufficiently discriminating gating strategies. With the right parameters and sufficient signal intensity, detecting and quantifying very rare events, even straight nanoparticle-protein/DNA-nanoparticle complexes, is feasible. Multiplexing of analytes can be performed by including other unique tags or even different sized nanoparticles in the complex. Furthermore, the real-time nature of flow cytometry makes it easier to break down the assay for better quality control of materials and detecting causes of non-specific binding.

An alternative way to analyze beads or tags is through capillary electrophoresis instead of flow cytometry. The concept is similar in that tags pass by an interrogation window in the capillary in single file, and are analyzed by laser-induced fluorescence measurement to decode the tags and quantify the captured target.

In one embodiment of the present invention, a method is provided for detecting for the presence of one or more target analytes (or biobarcodes) in a sample, each target analyte having at least two binding sites for specific binding interactions with specific binding complements, in a sample.

In another embodiment of the present invention, several different target analytes (or biobarcodes) may be detected, where each target analyte has at least two binding sites for specific binding interactions with specific binding complements, in a sample.

In another embodiment of the present invention, several kinds of particle beads and several kinds of nanoparticle probes may be used to allow detection of multiple target analytes or biobarcodes. For instance, linkers that bind to a first kind of analyte would also bind to a particular size nanoparticle, and a particle bead with a particular fluorescent marker attached. Different combinations of nanoparticles and particle bead/fluorescent markers will allow for the detection of various different target analytes in the same solution.

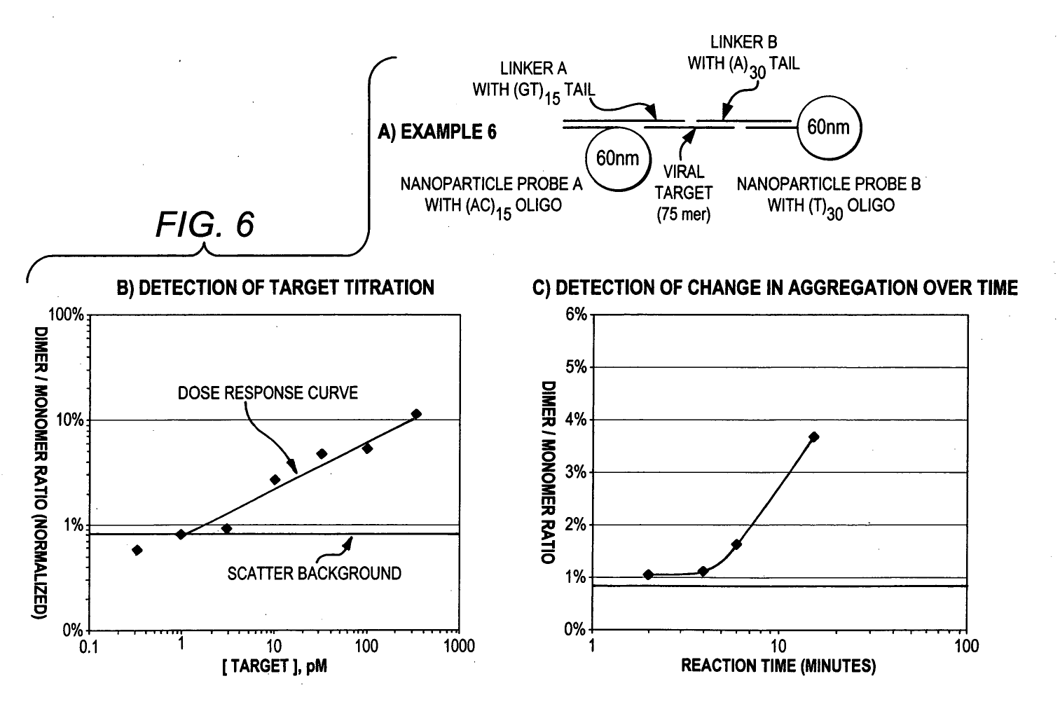

FIG. 7 provides an illustration of certain embodiments of the invention. FIG. 7 depicts the detection of a target molecule, said target having at least a first portion and a second portion The detection of the target is accomplished using two linker oligos (linker A and linker B), a nanoparticle probe A, and a nanoparticle probe B. Nanoparticle probe A is conjugated with at least one oligonucleotide sequence A. Linker A comprises at least a first portion and a second portion, said first portion comprising an oligonucleotide sequence A′ complementary to oligonucleotide sequence A, and said second portion comprising a sequence complementary to the first portion of the target. Nanoparticle probe B is conjugated with at least one oligonucleotide sequence B. Linker B comprises a first portion and a second portion, said first portion comprising an oligonucleotide sequence B′ complementary to the oligonucleotide sequence B, and said second portion comprising a sequence complementary to the second portion of the target.

### Examples

The following examples are offered to illustrate, but not to limit, the invention.

### Example 1

### Scatter Light Generated by Gold and Silver Nanoparticles in Flow Cytometry Assays

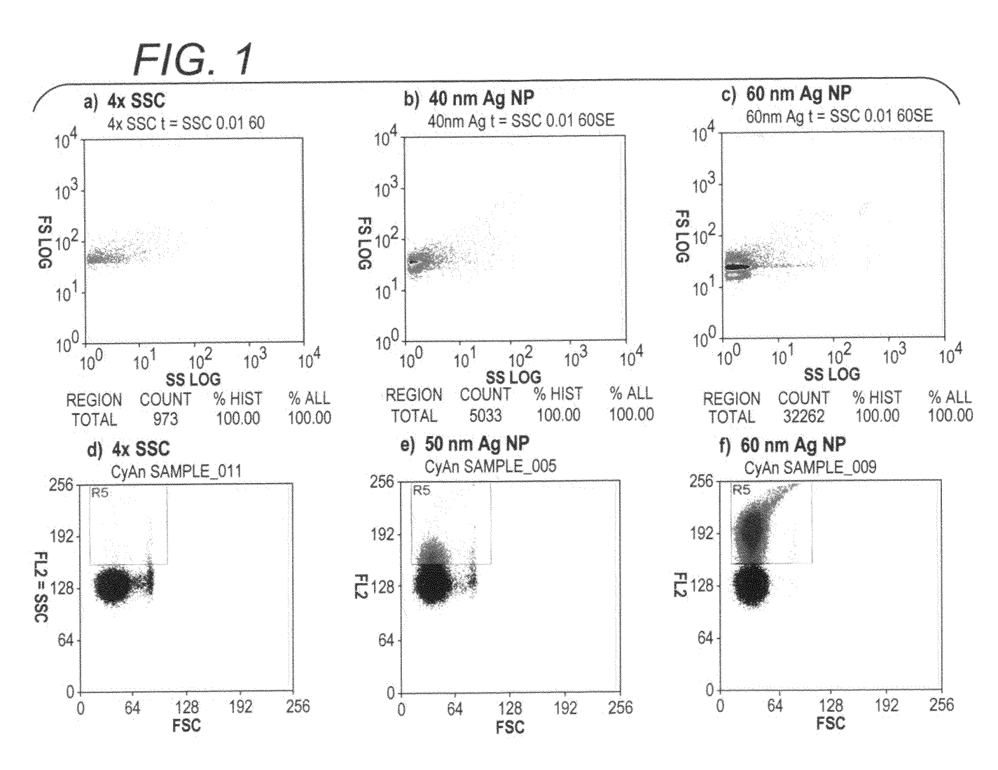

Gold and silver nanoparticles of various sizes were used to demonstrate the capability of nanoparticles to be used in flow cytometry assays. Using a Dako CytoMation 405 nm laser (Dako Denmark A/S, Glostrup, Denmark) or the Dako MoFlo 530 nm laser, forward and side scatter was adjusted to detect sub-micron particles. Gold and silver nanoparticles were obtained from BBInternational Ltd., Cardiff, UK. To demonstrate scatter light from 40 nm and 60 nm particles, 10⁶ Ag nanoparticles in 500 uL 4×SSC (Saline Sodium Citrate) were measured by side scatter in a 60 sec analysis (FIG. 1b-c), and 10⁶ Au nanoparticles in 500 uL 4×SSC were detected based on their red signal in a 60 second run (FIG. 1e-f), and were compared to measurement of 4×SSC alone (FIGS. 1a and 1d).

Nanoparticles of both types and sizes produced a bright and tight population, and larger nanoparticles produced more scatter. Aggregated nanoparticles might be the cause of the counts seen away from the main population.

Under the conditions used, 40 nm gold nanoparticle lack sufficient scatter intensity to be clearly separated from background. However, in further experiments, analyzing 30, 40, 50, 60 and 80 nm gold nanoparticle with excitation from either 535 nm or 635 nm lasers allowed us to resolve differences in relative scatter between the sols (data not shown). These results indicate that changes in side scatter intensity are sufficient to distinguish Nanoparticle size.

### Example 2

### Silver Amplification Induces Broad Side Scatter Shift of Gold Nanoparticles

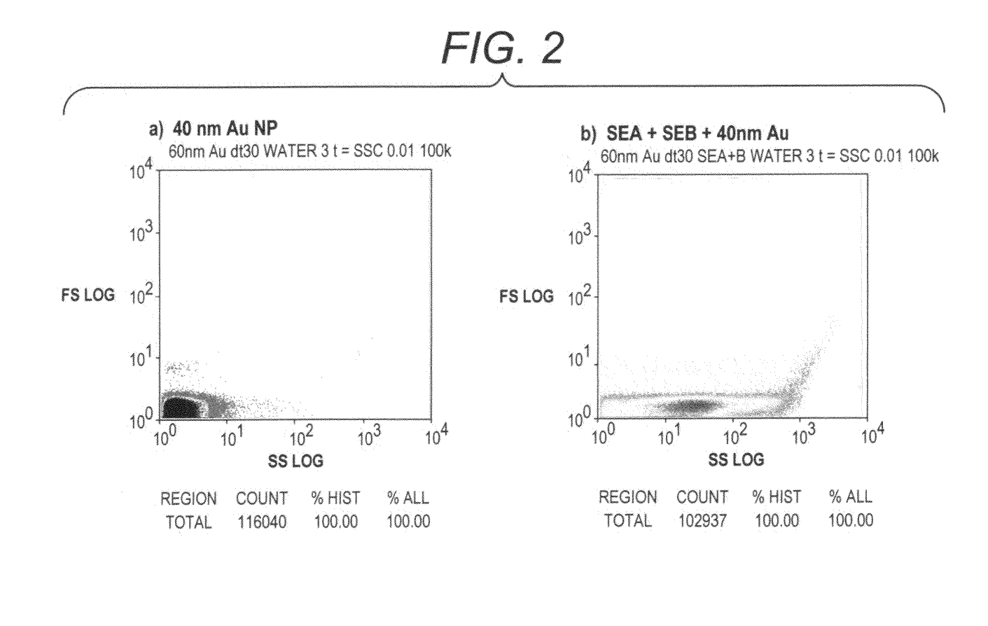

As shown in FIG. 2, silver staining of gold nanoparticles causes a large shift in side scatter and forward scatter, indicating a significant change in particle size and scatter properties. The experiments were conducted using 2 uL 40 nm gold nanoparticles were mixed with silver solution (2 uL Signal Enhancement A (SEA; Nanosphere, Northbrook, Ill.) and 2 uL Signal Enhancement B (SEB; These solutions are commercially available from Nanosphere, Northbrook, Ill. There are functionally equivalent commercially available Silver Enhancement reagents available (e.g. Silver Enhancement Solution A, #S-5020 and Silver Enhancement Solution B, #S-5145 Sigma-Aldrich, St. Louis, Mo.) and reacted for 5 minutes at room temperature. The reaction was stopped by diluting with 500 uL water. Scatter was detected with the CytoMation 405 nm laser. Silver-coating of gold nanoparticles caused a large shift in side scatter and a forward scatter tail (See FIG. 2b) indicative of significant changes in particle size and scatter properties.

### Example 3

### Silver Particles in Solution Detectable by Flow Cytometry

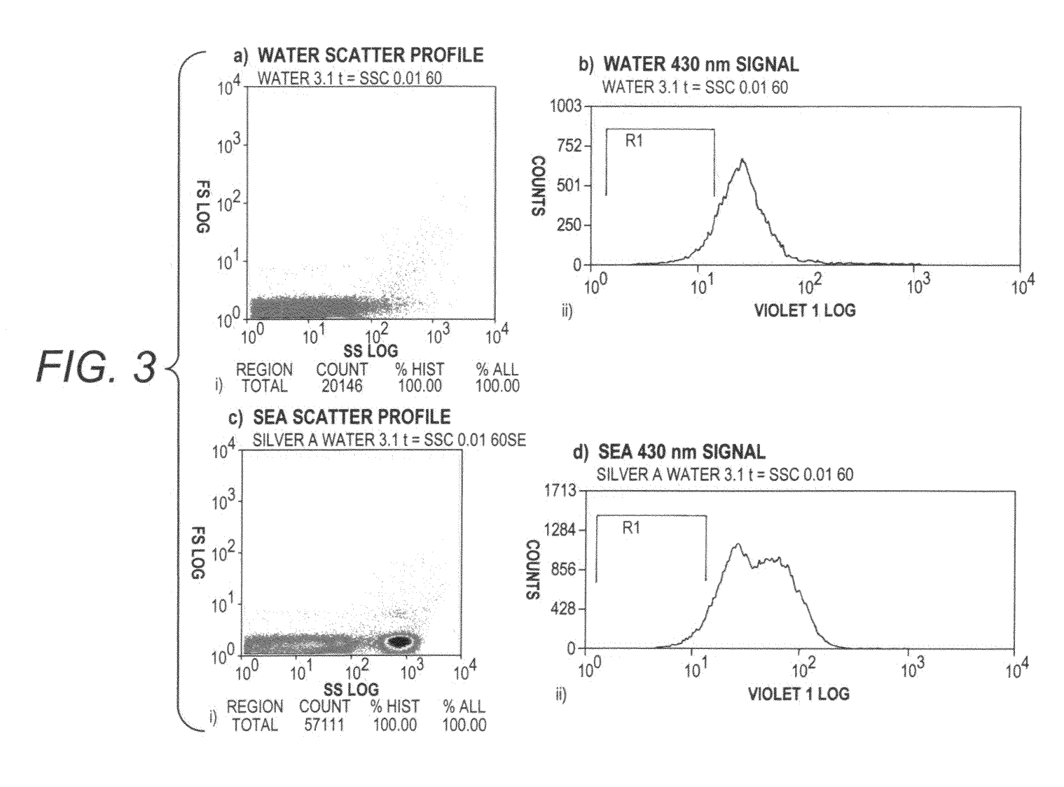

As shown in FIG. 3, Plasmon scatter light from silver particles can be seen by flow cytometry. A 100 uL aliquot of signal enhancement solution A (SEA) was transferred to a clear 1.5 mL tube. Due to opening of the box it was stored in, the SEA was briefly and randomly exposed to ambient light. Using the CytoMation 405 nm laser and 430 nm filter, silver particles induced by exposure to light were detected by side scatter (FIG. 3c). An increased 430 nm signal was also detected (FIG. 3d), indicating the plasmon scatter light from silver particles can be seen by flow cytometry.

### Example 4

### Aggregation of Nanoparticles Detectable Using Flow Cytometry

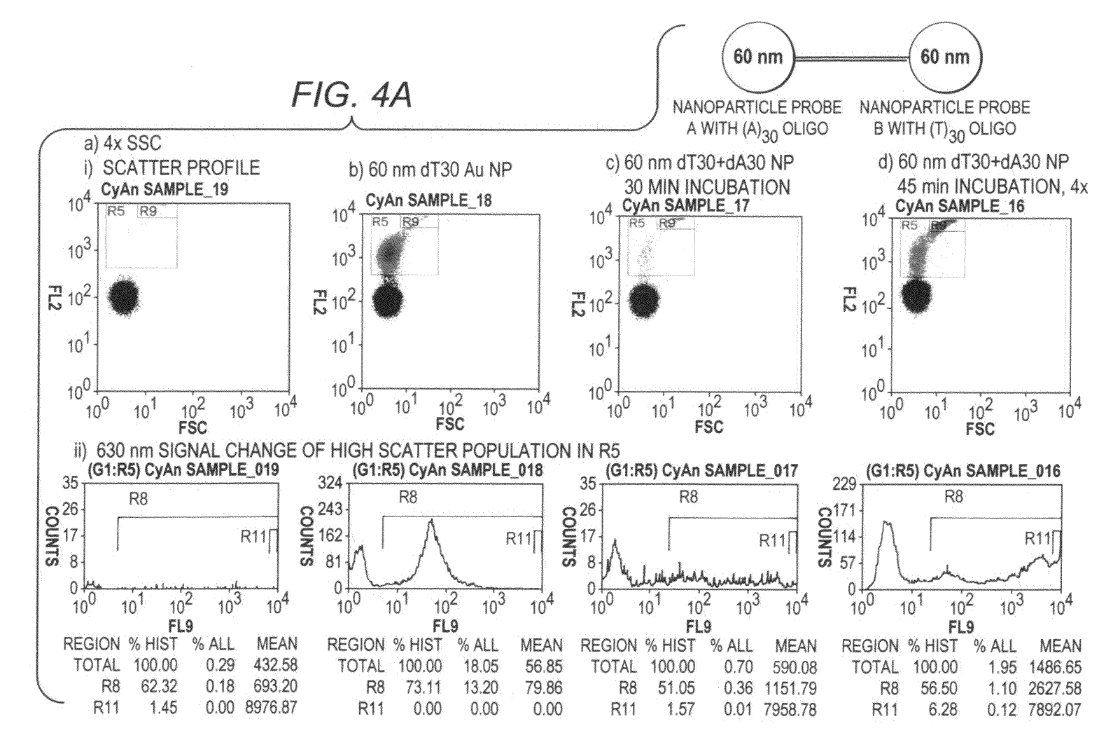

As shown in FIG. 4, complementary DNA-induced aggregation of nanoparticles showed a time dependent increase in 4×SSC and 630 nm intensity. 2 uL 509 pM 60 nm dT30 gold nanoparticles (Nanosphere, Northbrook, Ill.)+2 uL 210 pM 60 nm dA30 gold nanoparticles (Nanosphere, Northbrook, Ill.) were mixed in 26 uL 4×SSC/2% dextran sulfate (Sigma-Aldrich, St. Louis, Mo., Cat #D-8906) at room temperature. 2 uL of the mix were resuspended in 600 uL 4×SSC and analyzed with the Dako MoFlo (535 nm laser and 530 nm filter, 635 nm laser and 630 nm filter) after 30 minutes and 45 minutes. The complementary DNA-induced aggregation showed a time dependent increase in SSC and 630 nm intensity. The 630 nm scatter intensity of the aggregates were so intense as to begin to move off-scale. (See R12 in FIG. 4iii.)

### Example 5

### Target Analytes Detected Using Nanoparticle Probes and Flow Cytometry

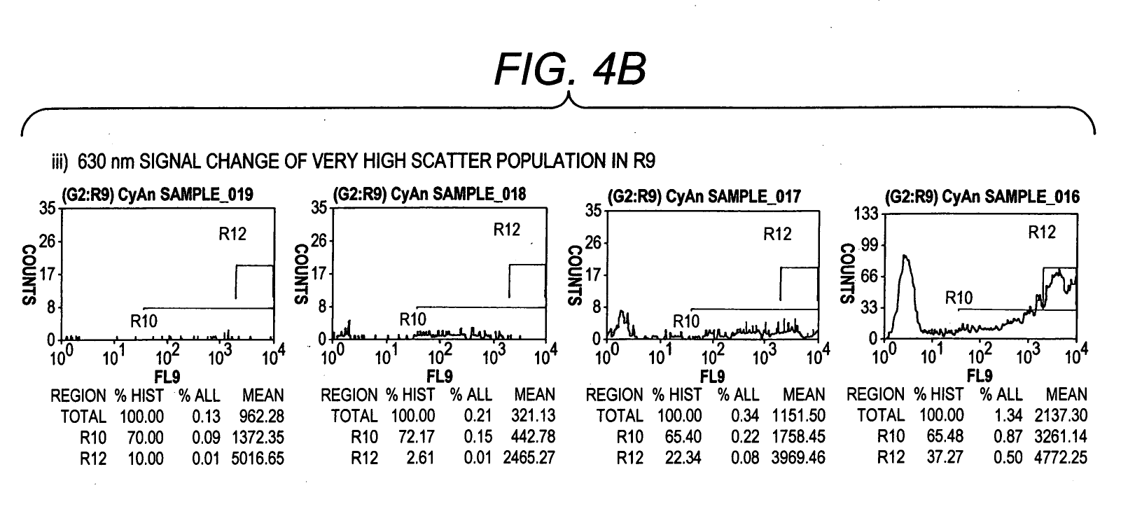

As shown in FIG. 5, increased amounts of dA80 target induced a new population with higher side scatter and 630 nm scatter, presumably dT30 dimers. 1 uL 509 pM 60 nm dT30 gold nanoparticles were mixed with increasing concentrations of dA˜80 target oligonucleotides (Biotin-BC1-dA30; Nanosphere, Northbrook, Ill.) in 20 uL 4×SSC/4% dextran sulfate and incubated overnight at room temperature. 4 uL of each mix was diluted in 400 uL 4×SSC. Light scatter was analyzed with the Dako MoFlo (535 nm laser and 530 nm filter, 635 nm laser and 630 nm filter), triggering on red signal.

### Example 6

### Viral Target Detected Using Nanoparticle Probes and Flow Cytometry

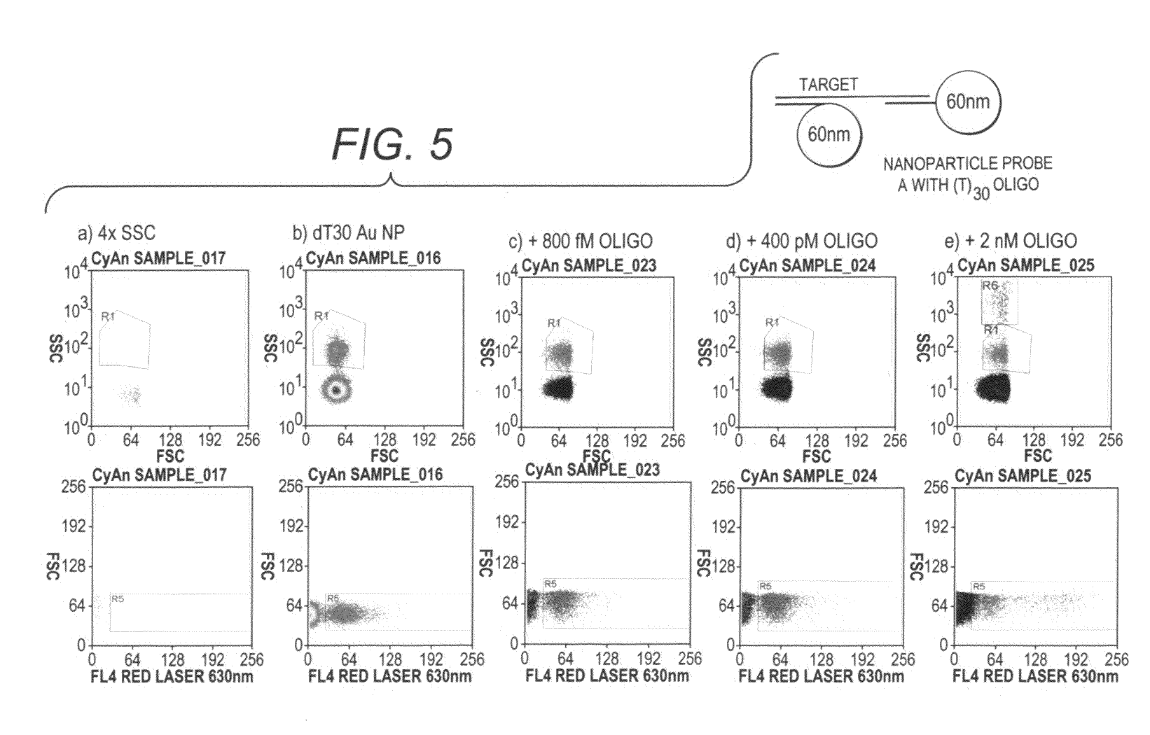

As shown in FIG. 6, detection of a viral target, said target having at least a first portion and a second portion, can be accomplished using nanoparticle probes. The detection of the target was accomplished using two linker oligos (linker A and linker B), a nanoparticle probe A, and a nanoparticle probe B (illustrated in FIG. 6A). Nanoparticle probe A is conjugated with at least one poly AC oligo. Linker oligo A comprises at least a first portion and a second portion, said first portion comprising a poly GT oligo (complementary to the poly-AC oligo conjugated to Nanoparticle probe A) and said second portion comprising a sequence complementary to the first portion of the viral target. Nanoparticle probe B is conjugated with at least one poly-T oligos. Linker oligo B comprises a first portion and a second portion, said first portion comprising a poly A oligo (complementary to the poly T oligo conjugated to Nanoparticle probe B), and said second portion comprising a sequence complementary to the second portion of the viral target.

To detect the viral target derived from the West Nile Virus genome, a complex (as illustrated in FIG. 6A) was formed by mixing an equimolar ratio of target oligo (5′-TGA CCA GTG CTA TCA ATC GGC GGA GCT CAA AAC AAA AGA AAA GAG GAG GAA AGA CCG GAA TTG CAG TCA TGA TTG-3′ SEQ ID NO: 1) and linker oligonucleotides (Linker Probe A: 5′-(GT)15-CAA TCA TGA CTG CAA TTC CGG TCT TTC CTC CTC TT-3′ SEQ ID NO: 2; Linker Probe B: 5-TTG AGC TCC GCC GAT TGA TAG CAC TGG TCA-(A)30 SEQ ID NO: 3; all synthesized by IDT, Coralville, Iowa) in 4×SSC (20×SSC (Ambion, Austin, Tex., cat #9770), diluted with DNA-grade water (Fisher Scientific, Pittsburgh, Pa., cat #BP2470-1) for 5 minutes at 80° C. The mixture was cooled to room temperature and serially diluted 3-fold. Then, 10 pM total nanoparticle probe mix were added in 20 uL 4×SSC/4% dextran sulfate (Sigma-Aldrich, D-8906, MW 500,000; St. Louis, Mo.). The mixture was incubated at room temperature for 2 hours. Detection was performed by flow cytometry. 1 uL of the sample was mixed in 400 uL 4×SSC and scatter light intensity at 630 nm was measured (FIG. 6B).

Another reaction mixture was formed by mixing 230 pM target complex in 8 pM of total nanoparticle probe mix in 20 uL 4×SSC/4% dextran sulfate. 1 uL of the sample was mixed in 400 uL 4×SSC and scatter light intensity at 630 nm was measured over time and the increase in dimer/monomer ratio was calculated (FIG. 6C).

### Example 7

### Scatter Intensity of Nanoparticles on MoFlow Cytometer Cersus Ocean Optics Spectrophotometer

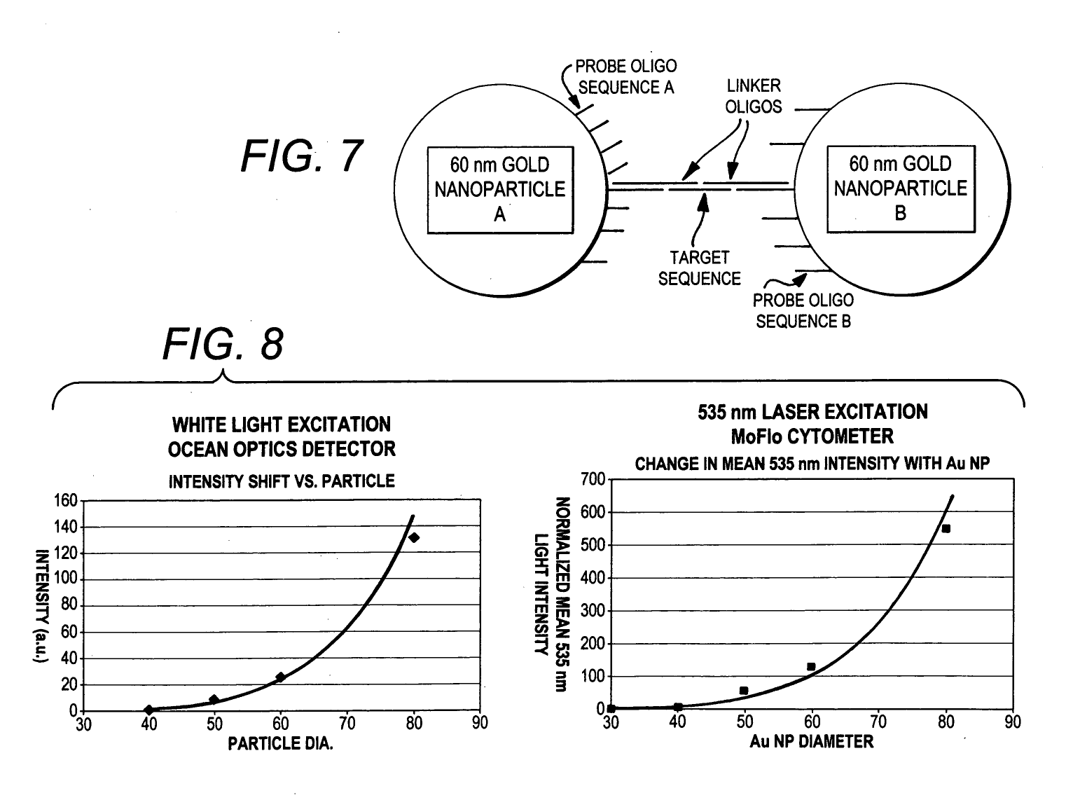

To determine if results discussed above using flow cytometry were consistent with results obtained with a spectrophotometer, the size-dependent scatter intensity of nanoparticles was determined on a MoFlow cytometer and an Ocean Optics spectrophotometer (Dunedin, Fla.). Various sizes of gold colloid (British Biocell International, Cardiff, UK) nanoparticles of various sizes (30 nm, 40 nm, 50 nm, 60 nm, and 80 nm) were diluted in phosphate-buffered saline (PBS) to 16 fM. The scatter intensity of these particle solutions were measured using an Ocean Optics spectrophotometer. Particle solutions were then analyzed on the MoFlo flow cytometer (run for 1 minute each). Detection was triggered by 488 nm signal and intensity measured at 535 nm. Intensity was normalized to levels over buffer background and plotted on a linear scale against the particle size. The dose response (relative signal intensity as a function of particle size) was identical on both instruments (FIG. 8). As shown in FIG. 8, the greater brightness of the 535 nm laser allowed 30 nm nanoparticles to be detected.

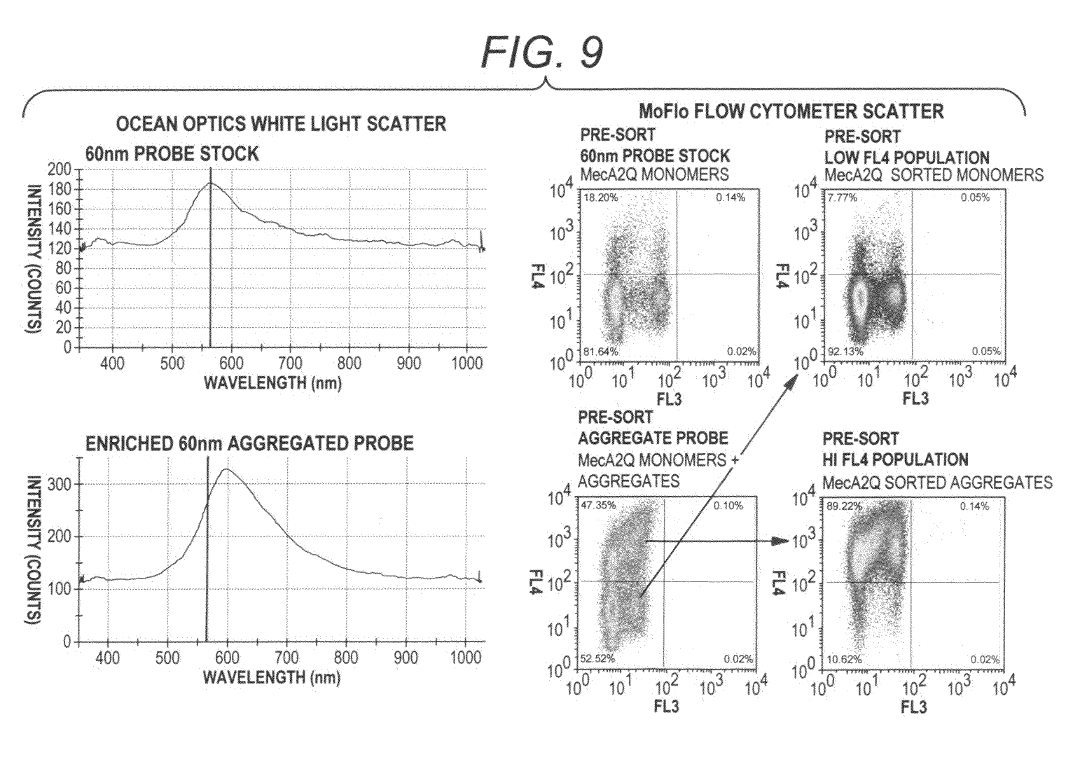

The scatter intensity of nanoparticle aggregates was also tested using the Ocean Optics spectrophotometer and compared to the intensity of aggregates measured by flow cytometry. 60 nm nanoparticle probes were aggregated using ionic conditions under which the surface charge leads to particle aggregation (incubation in 0.8 M NaCl). Aggregated probes were concentrated by centrifugation at 200×g for 15 min at RT. The scatter properties of singlet and aggregated nanoparticles were measured on an Ocean Optics spectrophotometer with white light illumination to show the shift in the resonance frequency. Particles and aggregates were then analyzed on a MoFlo flow cytometer (565 nm laser trigger and 635 nm detection wavelength) and sorted (fractionated) based on their scatter intensities (FL4 window). The sorted particle fractions were re-analyzed on the cytometer to check for purity and to establish that particle aggregates can be clearly identified and sorted by this method. As shown in FIG. 9, the enriched aggregate sample had an increased right-angle white light scatter detected by the spectrophotometer and an increased bright FL4 population detected by flow cytometry. The enriched aggregate sample was sorted based on FL4 intensity. Purity check showed they had sorted into two distinct populations.

### Example 8

Detection of DNA Target with 50 nm Probes and Protein Target with 30 nm Probes

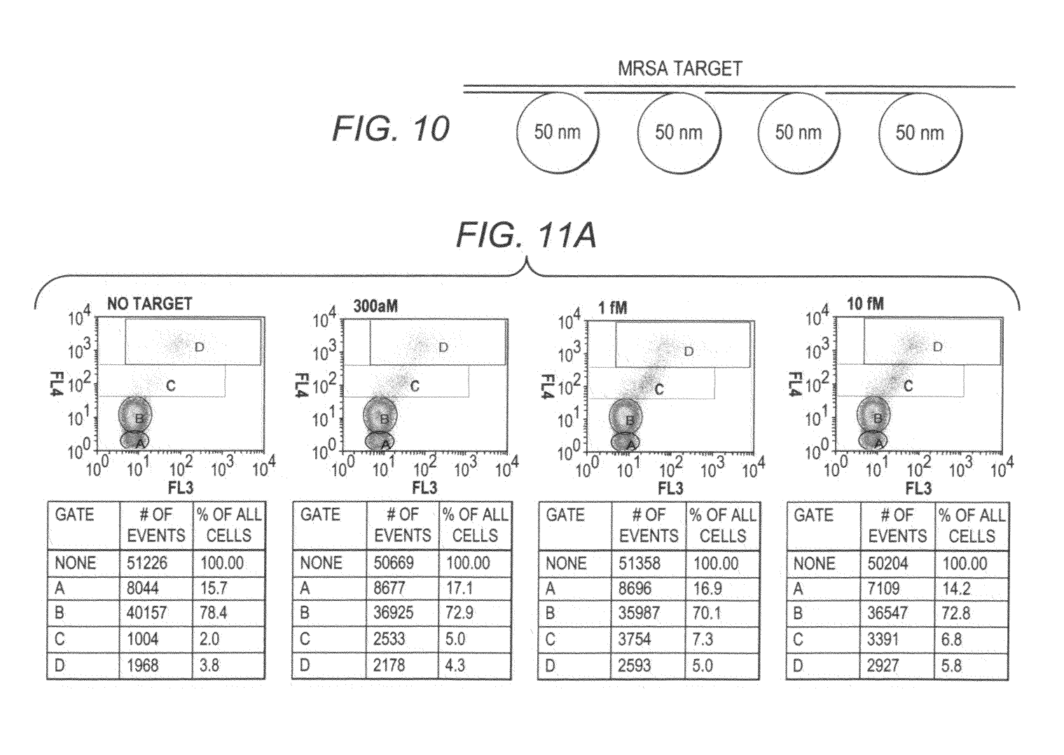

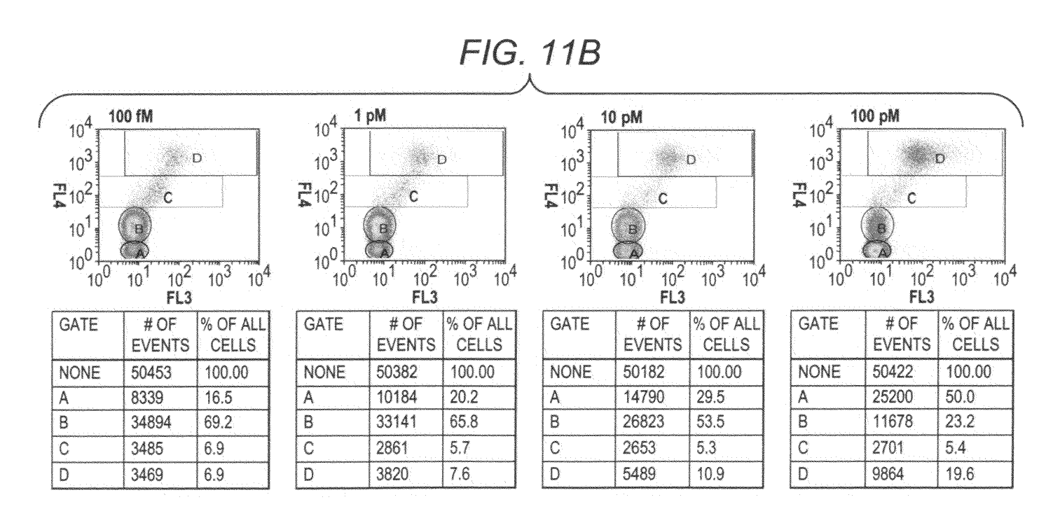

A MecA assay was designed to detect Methicillin resistant Staphylococcus aureus (MRSA) with four 50 nm nanoparticle probes as illustrated in FIG. 10. 10 pM total nanoparticle probes produced as previously described⁷¹ (2.5 pM each) in 70 uL 4×SSC/7.5% Formamide/4% dextran sulfate plus various concentrations of a 281 bp product derived by AmpliTaq PCR reaction from the MecA gene of MRSA (strain #700699 obtained from American Type Culture Collection, Manassas, Va.) were incubated at room temperature for 1.25 hours. For flow cytometry, 1 uL of hybridization reaction was mixed with 1 mL 4×SSC, and a 565 nm laser was used to detect 635 nm signal. The results are shown in FIG. 11.

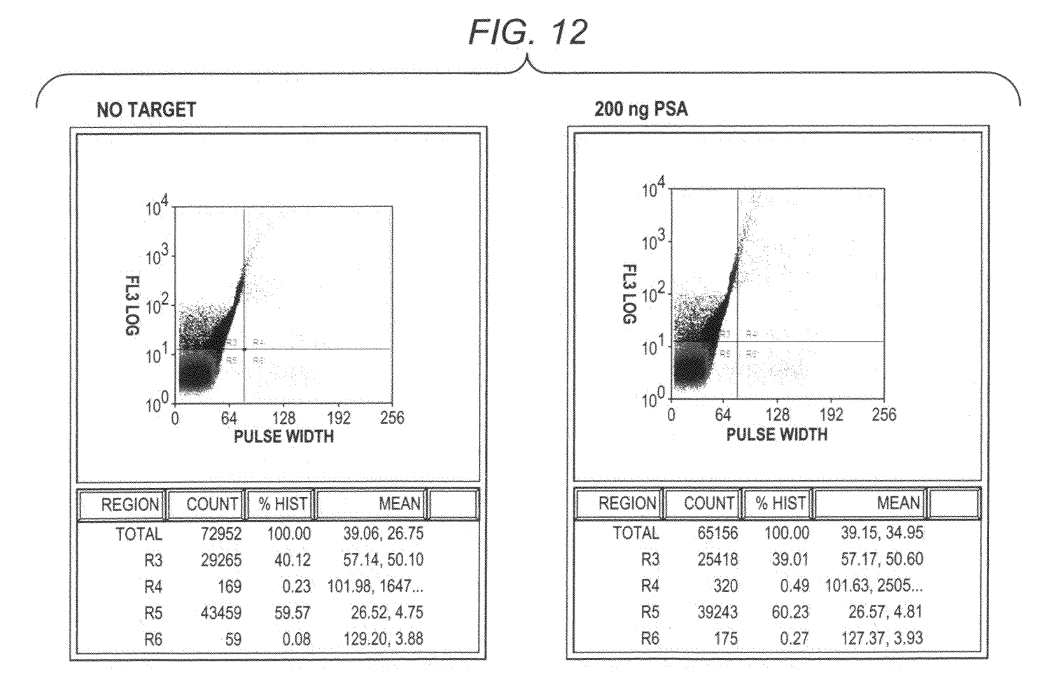

To demonstrate that nanoparticle probes can be used to detect protein targets, 10 pM of 30 nm nanoparticles co-loaded with anti-PSA polyclonal antibody (R&D Systems, Minneapolis, Minn.) (and non-specific oligonucleotides added for use in biobarcode assays) were incubated with or without 200 ng prostate specific antigen (OEM Concepts, Toms River, N.J., Cat #H6M07-323) in PBS for 30 minutes at room temperature. An aliquot of each sample was diluted to 1 pM in PBS. The samples were run on the MoFlo flow cytometer for 1 minute, analyzed using a 565 nm signal and discriminated by pulse width. An increase in events brighter in FL3 and/or greater pulse width was detected, indicative of aggregated probe (FIG. 12).

It should be understood that the foregoing disclosure emphasizes certain specific embodiments of the invention and that all modifications or alternatives equivalent thereto are within the spirit and scope of the invention as set forth in the appended claims.

### REFERENCES

(1) A. Pandey and M. Mann, Nature 2000, 405, 837-846.
(2) S. Fields and O. K. Song, Nature 1989, 340, 245-246.
(3) M. Ijksma, B. Kamp, J. C. Hoogvliet, W. P. van Bennekom, Anal. Chem. 2001, 73, 901-907.
(4) R. F. Service, Science, 2000, 287, 2136-2138.
(5) H. Zole, Monoclonal Antibodies, Springer-Verlag, New York, 2000, p.1-5.
(6) J. E. Butler, J. Immunoassay, 2000, 21(2 & 3), 165-209.
(7) P. Herbrink, A. Noorduyn, W. C. Van Dijk, Tech. Diagn. Pathol. 1991, 2, 1-19.
(8) C. A. Mirkin, R. L. Letsinger, R. C. Mucic, J. J. Storhoff, Nature 1996, 382, 607-609
(9) J. J. Storhoff, C. A. Mirkin, Chem. Rev. 1999, 99, 1849-1862.
(10) S.-J. Park, A. A. Lazarides, C. A. Mirkin, P. W. Brazis, C. R. Kannewurf, R. L. Letsinger, Angew. Chem. Int. Ed. 2000, 39, 3845-3848.
(11) T. A. Taton, C. A. Mirkin, and R. L. Letsinger, Science 2000, 289, 1757-1760.
(12) S.-J. Park, A. A. Lazarides, C. A. Mirkin, and R. L. Letsinger, Angew. Chem. Int. Ed. 2001, 40, 2909-2912.
(13) Z. Eshhar, M. Ofarim, and T. Waks, J. Immunol. 1980, 124, 775-780.
(14) M. Wilcheck and E. A. Bayer, Immunol. Today 1984, 5, 39-43
(15) N. Winssinger, J. L. Harris, B. J. Backes, P. G. Schultz, Agnew. Chem. Int. Ed. 2001, 40,
(16) G. MacBeath, A. N. Koehler, S. L. Schreiber, J. Am. Chem. Soc. 1999, 121, 7967-7968.
(17) P. J. Hergenrother, K. M. Depew, S. L. Schreiber, J. Am. Chem. Soc. 2000, 122, 7849-7850.
(18) J. J. Storhoff, R. Elghanian, R. C. Mucic, C. A. Mirkin, R. L. Letsinger, J. Am. Chem. Soc. 1998, 120, 1959-1964.
(19) R. C. Mucic, J. J. Storhoff, C. A. Mirkin, R. L. Letsinger, J. Am. Chem. Soc. 1998, 120, 12674-12675.
(20) L. M. Demers, C. A. Mirkin, R. C. Mucic, R. A. Reynolds III, R. L. Letsinger, R. Elghanian, G. Viswanadham, Anal. Chem. 2000, 72, 5535-5541.
(21) T. Brown, D. J. S. Brown, in Oligonucleotides and Analogues (Ed.: F. Eckstein), Oxford University Press, New York, 1991.
(22) L. A. Chrisey, G. U. Lee, and C. E. O'Ferral, Nucl. Acids. Res. 1996, 24, 3031-3039.
(23) Nicewamer-Pena, S. R. Freeman, R. G.; Reiss, B. D.; He, L.; Pena, D. J.; Walton, I. D.; Cromer, R.; Keating, C. D.; Natan M. J. Science 2001, 294, 137.
(24) Ferguson, J. A.; Steemers, F. J.; Walt, D. R. Anal. Chem. 2000, 72, 5618.
(25) Han, M.; Gao, X.; Nie, S. Nature biotech. 2001, 19, 631.
(26) R. K. Saiki et al., Science 1985, 230, 1350.
(27) R. K. Saiki et al., Science 1988, 239, 487.
(28) R. A. Gibbs, Curr. Opin. Biotechnol. 1991, 2, 69.
(29) S. Bortolin, T. K. Christopoulos, M. Verhaegen, Anal. Chem. 1996, 68, 834.
(30) B. Deiman, P. van Aarle, P. Sillekens, Mol. Biotechnol. 2002, 20, 163.
(31) S. E. Stiriba, H. Frey, R. Haag, Angew. Chem. Int. Ed. 2002, 41, 1329.
(32) S. A. Bustin, Journal of Molecular Endocrinology 2002, 29, 23.
(33) G. MacBeath, S. L. Schreiber, Science 2000, 289, 1760.
(34) H. Zhu et al., Science 2001, 293, 2101.
(35) B. B. Haab, M. J. Dunham, P. O. Brown, Genome Biol. 2001, 2(2): RESEARCH 0004.1.
(36) J.-M. Nam, S.-J. Park, C. A. Mirkin, J. Am. Chem. Soc. 2002, 124, 3820.
(37) T. Sano, C. L. Smith, C. R. Cantor, Science 1992, 258, 120.
(38) V. Ruzicka, W. Marz, A. Russ, W. Gross, Science 1993, 260, 698.
(39) A. McKie, D. Samuel, B. Cohen, N. A. Saunders, J. Immunol. Methods 2002, 270, 135.
(40) H. Zhou, R. J. Fisher, T. S. Papas, Nucl. Acids Res. 1993, 21, 6038.
(41) E. R. Hendrickson, T. M. Hatfield-Truby, R. D. Joerger, W. R. Majarian, R. C. Ebersole, Nucl. Acids Res. 1995, 23, 522.
(42) C. M. Niemeyer et al., Nucl. Acids Res. 1999, 27, 4553.
(43) B. Schweitzer et al., Proc. Natl. Acad. Sci. U.S.A. 2000, 97, 10113.
(44) C. M. Neimeyer, R. Wacker, M. Adler, Angew. Chem. Int. Ed. 2002, 40, 3169.
(45) C. M. Niemeyer, Trends Biotechnol. 2002, 20, 395.
(46) H. Yu, E. P. Diamandis, A. F. Prestigiacomo, T. A. Stamey, Clin. Chem. 1995, 41, 430.
(47) W. J. Catalona, et al., J. Am. Med. Assoc. 1995, 274, 1214.
(48) J. L. Stanford, et al., Prostate Cancer Trends 1973-1995, SEER Program, National Cancer Institute. NIH Pub. No. 99-4543. Besthesda, Md., 1999.
(49) J. Moul, J. Urol. 2000, 163, 1632.
(50) L. A. Chrisey, G. U. Lee, C. E. Oferral, Nucl. Acids Res. 1996, 24, 3031.
(51) Y. C. Cao, R. Jin, C. A. Mirkin, Science 2002, 297, 1536-1540.
(52) R. K. Saiki et al., Science 230, 1350 (1985).
(53) S. A. Bustin, Journal of Molecular Endocrinology 29, 23 (2002).
(54) M. U. Kopp, A. J. de Mello, A. Manz, Science 280, 1046 (1998).
(55) T. H. Rider et al., Science 301, 213 (2003).
(56) Y. W. Tang, G. W. Procop, D. H. Pershing. Clin. Chem. 43, 2021 (1997).
(57) G. M. Makrigiorgos, S. Chakrabarti, Y. Zhang, M. Kaur, B. D. Price, Nat. Biotechnol. 20, 936 (2002).
(58) W. P. Halford, Nat. Biotechnol. 17, 835 (1999).
(59) R. Sutthent et al., J. Clin. Microbiol. 41, 1016 (2003).
(60) B. Schweitzer, S. Kingsmore, Curr. Opin. Biotechnol. 12, 21 (2001).
(61) S. R. Nicewamer-Pena et al., Science 294, 137 (2001).
(62) M. Han, X. Gao, S. Nie, Nat. Biotechnol. 19, 631 (2001).
(63) X. Zhao, R. Tapec-Dytioco, W. Tan, J. Am. Chem. Soc. 125, 11474 (2003).
(64) L. A. Lortie et al., J. Clin. Microbiol. 29, 2250 (1991).
(65) L. R. Zeph, X. Y. Lin, G. Stotzky, Curr. Microbiol. 22. 79 (1991).
(66) C. J. Yu et al., J. Am. Chem. Soc., 123, 11155 (2001).
(67) T. A. Taton, C. A. Mirkin, R. L. Letsinger, Science 289, 1757 (2000).
(68) S.-J. Park, T. A. Taton, C. A. Mirkin, Science 295, 1503 (2002).
(69) Y. C. Cao, R. Jin, C. A. Mirkin, Science 297, 1536 (2002).
(70) A. Saghatelian, K. M. Guckian, D. A. Thayer, M. R. Ghadiri, J. Am. Chem. Soc. 125, 344 (2003).
(71) Storhoff, J. J., A. D. Lucas, et al. (2004). “Homogeneous detection of unamplified genomic DNA sequences based on calorimetric scatter of gold nanoparticle probes.” Nat Biotechnol 22(7): 883-7.
(72) Sonnichsen, C., B. M. Reinhard, et al. (2005). “A molecular ruler based on plasmon coupling of single gold and silver nanoparticles.” Nat Biotechnol 23(6): 741-5.
(73) Yguerabide, J. and E. E. Yguerabide (1998). “Light-scattering submicroscopic particles as highly fluorescent analogs and their use as tracer labels in clinical and biological applications.” Anal Biochem 262(2): 157-76.
(74) Yguerabide, J. and E. E. Yguerabide (1998). “Light-scattering submicroscopic particles as highly fluorescent analogs and their use as tracer labels in clinical and biological applications.” Anal Biochem 262(2): 137-56.
(75) Bohmer, R. M. and N. J. King (1984). “Flow cytometric analysis of immunogold cell surface label.” Cytometry 5(5): 543-6
(76) Festin, R., B. Bjorklund, et al. (1987). “Detection of triple antibody-binding lymphocytes in standard single laser flow cytometry using colloidal gold, fluorescein and phycoerythrin as labels.” J Immunol Methods 101(1): 23-8.
(77) Siiman, O., K. Gordon, et al. (2000). “Immunophenotyping using gold or silver nanoparticle-polystyrene bead conjugates with multiple light scatter.” Cytometry 41(4): 298-307.
(78) Goix, P, 2006. Single Molecule “Flow Immunoassay” Detection: Repurposing Existing Marker For Clinical Validation. CHI Clinical Biomarker Summit Presentation, San Diego.
(79) Elghanian, R. , Storhoff, J. J. et al. , 1997. Selective colorimetric detection of polynucleotides based on the distance-dependent optical properties of gold nanoparticles. Science 277: 1078-81

What is claimed is:

1. A method for detecting for the presence of one or more target analytes in a sample comprising the steps of:
a) providing a plurality of nanoparticle probes conjugated to binding moieties capable of binding to a first binding site of the target analyte, wherein the nanoparticle probes comprise a metallic material and have an average diameter of less than 200 nanometers;
b) providing a capture surface comprising binding moieties capable of binding to a second binding site of the target analyte;
c) contacting the nanoparticle probes and capture surface with a sample believed to contain target analytes under conditions effective to allow for binding of the target analyte with the nanoparticle probes and the capture surface to form a complex in the presence of the target analyte;
d) optionally washing the capture surface containing the complex formed in (c) to remove all non-bound nanoparticle probes;
e) releasing the captured nanoparticle probes from the capture surface;
f) subjecting the released nanoparticle probes to confinement conditions under which individual nanoparticle probes can be detected;
g) irradiating nanoparticle probes in the confinement conditions with a light beam; and
h) measuring scatter light generated in step (g) to determine the number of released nanoparticle probes as an indicator of the presence of target analyte in the sample.

2. A method for detecting for the presence of one or more target analytes in a sample comprising the steps of:
a) providing a first nanoparticle probe conjugated to binding moieties capable of binding to a first binding site of the target analyte, wherein the nanoparticle probes comprise a metallic material and have an average diameter of less than 200 nanometers;
b) providing a second nanoparticle probe conjugated to binding moieties capable of binding to a second binding site of the target analyte, wherein the nanoparticle probes comprise a metallic material and have an average diameter of less than 200 nanometers;
c) contacting the first and second nanoparticle probes with a sample believed to contain target analytes under conditions effective to allow for binding of the target analyte with the binding moieties on the first and second nanoparticle probes to form a complex in the presence of the target analyte;
d) subjecting the sample-nanoparticle probe mixture in (c) to confinement conditions under which individual nanoparticle probes or nanoparticle probe-target complexes can be detected;
e) irradiating the complex with light of a frequency range that covers the plasmon resonance frequency of the nanoparticles; and
f) measuring scatter light frequency to differentiate single from complexed nanoparticles,
whereby the presence of complexed particles is indicative of the presence of the target analyte in the sample.

3. The method of claim 1 or 2, wherein the nanoparticle probes bind to the target analyte indirectly via specific linker molecules.

4. The method of claim 1 or 2, wherein the binding moieties comprise oligonucleotides, antibodies, aptamers, or some combination thereof.

5. The method of claim 1 or 2, wherein the nanoparticle probes are about 30 to about 150 nm in diameter.

6. The method of claim 1 or 2, wherein the target analyte is a protein or hapten and the binding moieties are antibodies.

7. The method of claim 6, wherein the antibodies are polyclonal antibodies or monoclonal antibodies.

8. The method of claim 1 or 2, wherein the target analyte is a sequence from a genomic DNA sample and the binding moieties are oligonucleotides, the oligonucleotides having a sequence that is complementary to at least a portion of the genomic sequence.

9. The method of claim 8, wherein the genomic DNA is eukaryotic, bacterial, fungal or viral DNA.

10. The method of claim 1 or 2, wherein the target analyte is a sequence from episomal DNA sample and the binding moieties are oligonucleotides, the oligonucleotides having a sequence that is complementary to at least a portion of the episomal DNA sequence.

11. The method of claim 1 or 2, wherein the confinement conditions are generated by flow cytometry.

12. The method of claim 1 or 2, wherein the confinement conditions are generated by capillary eletrophoresis.

13. The method of claim 1 or 2, wherein at least one of the nanoparticle probes is further labeled with a Raman active group.

14. The method of claim 13, wherein more than one type of Raman active group is used in equal or different concentrations.

15. The method of claim 1 or 2, wherein the nanoparticle probes comprise gold, silver, copper, or platinum.

16. The method of claim 1 or 2, wherein the nanoparticle probes are core-shell nanoparticles.

17. The method of claim 1, wherein the target analyte binds to the capture surface indirectly via specific linker molecules.

18. The method of claim 1, wherein the capture surface is a microtiter well.

19. The method of claim 1, wherein the capture surface containing the complex formed in (c) is isolated from all non-bound nanoparticle probes.

20. The method of claim 19, wherein the capture surface is a magnetic bead.

21. The method of claim 1, wherein the plurality of nanoparticle probes comprises nanoparticle probes of different shapes, each differently shaped nanoparticle probe being conjugated to binding moieties that bind to a different target analyte, and wherein each differently shaped nanoparticle probe creates unique scatter light when irradiated, thereby indicating the presence of the target analyte to which it binds.

22. The method of claim 1, wherein the plurality of nanoparticle probes comprises nanoparticle probes of different materials, each nanoparticle probe of different material being conjugated to binding moieties that bind to a different target analyte, and wherein each nanoparticle probe of different material creates unique scatter light when irradiated, thereby indicating the presence of the target analyte to which it binds.

23. The method of claim 1, wherein the plurality of nanoparticle probes comprises nanoparticle probes of different sizes, each nanoparticle probe of different size being conjugated to binding moieties that bind to a different target analyte, and wherein each nanoparticle probe of different size creates unique scatter light when irradiated, thereby indicating the presence of the target analyte to which it binds.

24. The method of claim 1 or 2 further comprising a step of providing one or more labeled microbeads that can bind to either the target analyte or to a nanoparticle probe, thereby capable of forming a complex with the nanoparticle probes and the target analyte.

25. The method of claim 24, wherein at least one microbead is fluorescently labeled.
